(19) United States
(12) Patent Application Publication
(10) Pub. No.: US 2023/0233402 A1
(43) Pub. Date: Jul. 27, 2023

(54) FACE WORKOUT AND REJUVENATOR WAND

(71) Applicant: Sandra Hernandez-Black, Chula Vista, CA (US)

(72) Inventor: Sandra Hernandez-Black, Chula Vista, CA (US)

(21) Appl. No.: 17/582,366

(22) Filed: Jan. 24, 2022

(51) Int. Cl. A61H 23/06; A61K 8/34; A61K 8/67; A61K 8/68; A61K 8/73; A61K 8/9789; A61Q 19/08

(52) U.S. Cl. CPC A61H 23/06; A61K 8/347; A61K 8/675; A61K 8/676; A61K 8/678; A61K 8/68; A61K 8/735; A61K 8/9789; A61K 8/67; A61Q 19/08; A61H 2201/0153; A61H 2201/0214; A61H 2205/022; A61H 2201/169

(57) ABSTRACT

A hand-held device with a paddle, an arm and a handle. Using the handle and arm, the paddle is slapped/tapped against the face to, in effect, provide a workout for an individual's facial skin and facial muscles. One purpose of a workout for the facial muscles is to tighten the facial muscles by inducing/stimulating the production of new and healthy collagen, improve skin firmness and texture, and blood circulation. The skin's cells need good circulation to heal and produce collagen. The slapping/tapping of the face creates a superficial trauma to the skin and muscles which tricks the body into producing high levels of collagen to assist in the healing process without causing any actual damage to the skin.

### FIELD OF THE INVENTION

The invention is directed to a device formed by a paddle with attached arm and handle for use as a face workout and rejuvenator wand.

### BACKGROUND

As facial skin ages, it tends to become less elastic and prone to wrinkle. Creams, lotions and serums are well known for being applied to the face in an attempt to provide more elasticity and reduce wrinkles. While such creams, lotions and serums may create a short term improvement by adding moisture to the skin, they must be applied on a regular basis and only provide minimal, if any, improvement. However, there is no cream, lotion or serum currently available in the market that would strengthen and tighten the facial muscles. Cosmetic surgery such as laser treatments is an invasive technique which is sometimes used to reduce facial wrinkles. While long lasting, such surgery is expensive and can require many days for the skin to fully heal. Small handheld devices, referred to as ultrasonic spatulas, use a thin blade which vibrates at ultrasonic speeds for exfoliating and cleaning by scraping facial skin with the thin blade. Although less invasive than cosmetic surgery, such ultrasonic spatulas can result in unwanted irritation and are more of an exfoliator as opposed to a treatment to reduce wrinkles, strengthen the facial muscles and the like. Other treatments include injecting products such as Botox® which reduces muscle activity at the injection site which can result in a smoothing of wrinkles, but treatments need to be repeated on a regular basis and tend to be expensive.

Thus, there is a need for an easy to use, inexpensive device which provides much better results than can be obtained using skin creams, lotions and serums, without the need for surgery or other invasive treatments and which provides better results than can be obtained with ultrasonic spatulas.

### SUMMARY OF THE INVENTION

The invention is a simple hand-held device with a paddle, an arm and a handle. Using the handle and arm, the paddle is slapped/tapped against the face to, in effect, provide a workout for an individual's facial muscles. One purpose of a workout for the facial muscles is to tighten the facial muscles which provides the following benefits:
Production of high levels of collagen by inducing/stimulating the production of new and healthy collagen.
Improvement of skin firmness and texture
Stimulation of blood circulation
Natural face glow
Strengthening of the skin
Stimulus of chemoreceptors, thermoreceptors and mechanoreceptors for an effective facial workout and a great aid on the conduction and absorption of nutrients applied to the skin
Strengthening of the neck muscles—This is a side effect due to pressure applied to the face to tighten the face muscles. The neck muscles need to hold a contracted position in order to apply resistance to the slapping/tapping of the face

The skin's cells need good circulation to heal and produce collagen. By slapping/tapping the face with the invented rejuvenator wand, the facial skin is improved by stimulating blood circulation, collagen production and giving the skin a natural glow. This happens because the slapping//tapping of the face creates a superficial trauma to the skin and muscles which tricks the body into producing high levels of collagen to assist in the healing process without causing any actual damage to the skin. Although it is difficult to develop muscles at an older age, it is not impossible if a workout is done consistently. However, the sooner one starts working out the face muscles in life, the easier it would be to maintain healthy, defined and strong face muscles. The rejuvenator wand, is a great innovative tool to help to develop and maintain the face muscles.

### BRIEF DESCRIPTION OF THE DRAWINGS

FIG. 1 is perspective view showing the invented face workout and rejuvenator wand.

FIG. 2 is a cross-section of the wand taken along line 2-2 of FIG. 1.

### DETAILED DESCRIPTION OF THE INVENTION

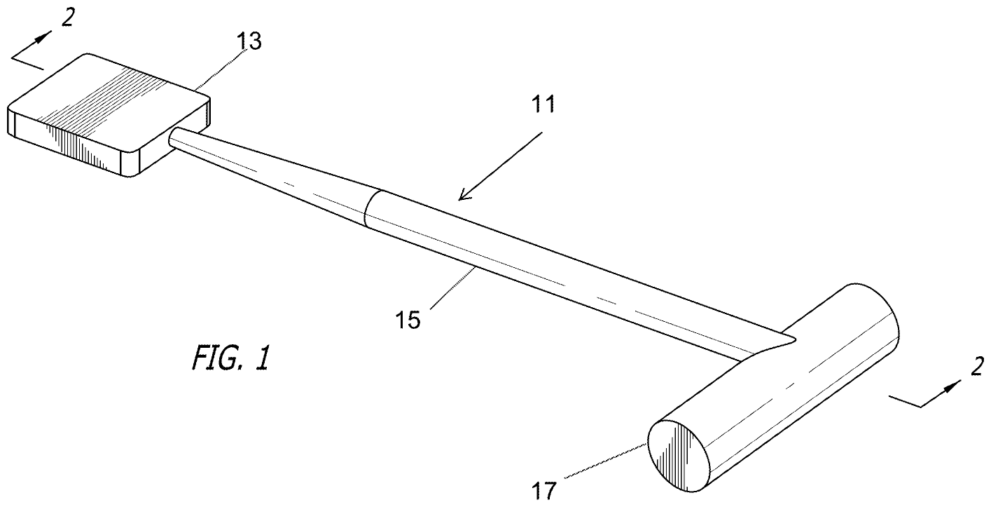

With reference to FIG. 1, the rejuvenator wand 11 has three main components, namely a paddle 13, an arm 15 connected to the paddle and a handle 17 connected to the arm.

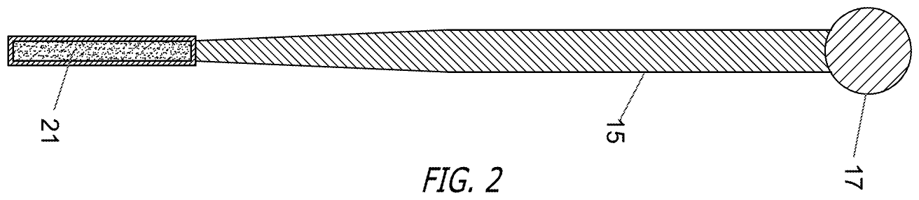

The paddle has two main components, namely an outside portion and, a preferably hollow interior 21 as shown in FIG. 2.

The outside portion preferably is made of a soft silicone material. The silicone is manufactured in a manner to achieve an optimal firmness and texture. The optimal firmness would be one where the paddle 13 flexes when it is pressed against a surface using a medium force as would be applied by a person of average strength, and returns to its original shape/place when the force is removed. In an embodiment, the material of the paddle has a firmness of Shore A 30 to Shore A 40. The texture of the outside surface of the paddle should be smooth so that when pressed against the face, the paddle does not impart a pattern corresponding to the surface of the paddle. The paddle may also be made with rubber or any other silicone like material to achieve an appropriate firmness/softness/texture for the paddle which provides a flexibility and texture as noted above.

In an embodiment, the hollow interior 21 is filled with a non-freezable gel so that when the paddle is put in the freezer, the surface of the paddle and the gel will get very cold. This is to give the users the additional benefit of a cold paddle so to not only have the benefit of a great face workout, but to help reduce puffiness around the eyes, reduce face swelling and inflammation in general, increase absorption of any serum, cream or lotion applied to the face, and many other benefits to the face. However, the hollow interior is optional and the paddle may also be solid, that is with no hollow interior. The manner of use is the same whether the paddle is solid or hollow and filled with gel, the only difference being the gel allows the paddle to be cool to the touch when placed in a freezer as described above. Even when the paddle is placed in a freezer for an extended period of time, the gel will get very cold, but will not freeze. Regardless of the paddle being use cold or not cold, the firmness of the paddle will not be affected.

Although the precise size of the paddle is not important, a typical paddle dimensions are length 1.5 to 2 inches, width 0.5 to 0.8 inches and height 2.2 to 2.5 inches.

The other two components of the wand, namely the arm, is connected to the bottom of the paddle and the handle is connected to the bottom of the arm.

The arm 15 connected to the bottom of the paddle should be relatively inflexible and made of a material such as stainless steel. A typical arm dimensions are 7 to 8 inches long and 0.5 to 0.7 inches in diameter. The handle 17 attaches to the arm as an ergonomic handle to provide an easy way to grip the wand. Typically, the dimensions of the handle are 2.5 to 3 inches long with a diameter of 0.50 to 1 inch. Preferably, the handle is made of soft, rubber like material.

The mechanisms for attaching the arm to the paddle and the handle to the arm are not important for an understanding of the invention and may be permanent or removable. For example, the parts can be glued together or connected by threaded engagement. The handle and arm can also be an integrated unit.

Preferably, serums, creams and/or lotions are applied to the face before and after uses of the wand. The rejuvenator wand work well without the below specific serums, but for optimal results, serums having the following ingredients should be applied.

Before use main serum ingredients include niacinamide, calendula, ceramides, galactomyces and cannabidiol (CBD). The main purpose of the before use serum is to add hydration and fortify the epidermis in preparation to a workout. The user does not have to use any serums or lotions, but it is preferable to use a serum that at least contains two of the above mentioned ingredients to achieve optimal results

After use, the main serums include vitamin C, vitamin E, vitamin D, hyaluronic acid, glycol and green tea. The main purpose of the after use serum is to aid healing and recuperation.

To obtain optimal results, the face rejuvenator wand should be used twice per day, preferably once in the morning and once at night.

### Use at Night:

Before going to bed and with a clean face, apply the pre-work out serum and immediately start working out the face from one minute to two minutes. One or two rejuvenator wands can be used. With only one rejuvenator wand, it might take a little bit longer since with two wands the right and left areas of the face can be worked on at the same time.

Grabbing the wand or wands from the handle, the wand should be moved back and forth like slapping/tapping the face type of movements. The slapping/tapping is done very mildly for the first two weeks. Then, after two weeks the intensity must be increased. Just like working out any other muscle in the body, it is preferable to start slow and then gradually increase the intensity.

The face workout areas are:
Forehead (right and left)—15 seconds each side.
Forehead (middle)—15 seconds
Upper cheeks—15 seconds each
Lower cheeks—15 seconds each
Jaw upper area—15 seconds each
Jaw lower area—15 seconds each

Once the workout is finished, the post workout serum should be applied and then, if desired the user can follow their normal beauty routine.

### Use in the Morning:

Starting with a flesh/clean face, apply the pre-work out serum and immediately start working out the face from one minute to two minutes. One or two rejuvenator wands can be used, but if only one rejuvenator wand is used, it might take a little bit longer since with two wands the user can work out the right and left areas of the face at the same time.

Grabbing the wand or wands from the handle, the wand should be moved back and forth with a slapping/tapping of the face type of movements. The slapping/tapping is done very mildly for the first two weeks. Then after two weeks the intensity should be increased. Just like working out any other muscle in the body, it is preferable to start slow and then gradually increase the intensity.

The face workout areas are:
Forehead (right and left)—15 seconds each side.
Forehead middle—15 seconds
Upper cheeks—15 seconds each
Lower cheeks—15 seconds each
Jaw upper area—15 seconds each
Jaw lower area—15 seconds each

Once the workout is finished, the post workout serum is preferably applied and then, if desired the user can follow their normal beauty routine.

As explained above, the user should start with mild slapping/tapping and then work their way up. After two months, the user should apply the highest intensity. The highest intensity is user dependent, but is typically reached when the user notices that with each stroke, the muscle tightens/contracts very firmly and the user achieves a sense of pleasurable pain, similar to the pain when any other body muscle is given a healthy workout.

Areas to avoid slapping/tapping are the eyes, under eye area, nose and lips.

The paddle portion of the rejuvenator wand can also be used to help de-puff the eyes. If the user has two paddles/wands, they can use them at the same time to put them on their eyes when they are cold to help to de-puff the eye. One or two paddles are placed on the eyes. The paddles are shaped to fit comfortably over the eyes.

Although the invention has been described in terms of specific embodiments, the scope of the invention is limited only by the following claims.

I claim:

1. A face workout and rejuvenator wand comprising:
a paddle for slapping/tapping facial skin;
an arm coupled to the paddle;
a handle coupled to the arm, the handle configured to be handheld so that a back and forth movement of the handle translates into back and forth movements of said paddle to provide said slapping/tapping of said facial skin to stimulate blood circulation, collagen production and give the facial skin a natural glow;
wherein said stimulation and collagen production are obtained by applying said paddle slapping/tapping to the facial skin to different areas of a face for predetermined periods of time.

2. The face workout and rejuvenator wand of claim 1 where said different areas of the face include right, middle and left forehead, upper cheeks, lower cheeks, upper jaw and lower jaw.

3. The face workout and rejuvenator wand of claim 2 where said predetermined periods of time are 15 seconds for each said different area.

4. The face workout and rejuvenator wand defined by claim 1 wherein the paddle is made of silicone.

5. The face workout and rejuvenator wand defined by claim 4 wherein the paddle made of silicone has a hollow interior filled with a non-freezable gel.

6. The face workout and rejuvenator wand defined by claim 1 wherein the material of the paddle has a firmness of Shore A 30 to Shore A 40.

7. A method for stimulating blood circulation and collagen production to facial skin and facial muscles by using a face workout and rejuvenator wand as defined by claim 1 comprising:
applying a serum or lotion with ingredients selected from the group consisting of niacinamide, calendula, ceramides, galactomyces and cannabidiol (CBD) to add hydration and fortify the epidermis to relax the facial skin and facial muscles;
after said applying, slapping/tapping said facial skin with said paddle to said different areas of said face for said predetermined period of time;
after said slapping/tapping said facial skin, apply a serum or lotion with ingredients selected from the group consisting of vitamin C, vitamin E, vitamin D, hyaluronic acid, glycol and green tea to aid healing and recuperation of said facial skin and muscles.

8. The method defined by claim 7 wherein said slapping/tapping said facial skin is to the:
right and left forehead for 15 seconds each side;
middle forehead for15 seconds,
upper cheeks for 15 seconds each side,
lower cheeks for 15 seconds each side,
upper jaw areas for 15 seconds each side,
lower jaw lower areas for 15 seconds each side.

9. The method defined by claim 7 wherein said slapping/tapping said facial skin is undertaken twice per day.
